(19) United States
(12) Patent Application Publication
(10) Pub. No.: US 2008/0027404 A1
(43) Pub. Date: Jan. 31, 2008

(54) Absorbent articles and functional wipes

(76) Inventors: Rosa Alejandra Hernandez, Cincinnati, OH (US); Ilonka Bibiana Laviz, Mason, OH (US); Irene Spalazzi, Mason, OH (US); Ronald Bosman Visscher, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/495,297

(22) Filed: Jul. 28, 2006

(51) Int. Cl. A61F 13/15

(52) U.S. Cl. 604/385.06; 604/385.02

(57) ABSTRACT

An absorbent article combination comprising an absorbent article and a wipe. The wipe can be a functional shape and/or in the form of a mitt. The wipe can be individually packaged and attached to an absorbent article that can be individually packaged.

### FIELD OF THE INVENTION

The present invention relates generally to the field of disposable absorbent articles and wipes.

### BACKGROUND OF THE INVENTION

Absorbent articles, such as sanitary napkins, liners, and tampons, are generally used by women to absorb menstrual or other body exudates. Diapers and incontinence articles are used by babies or other incontinent individuals to absorb and contain body exudates, such as urine and/or feces. Often, the use and changing of these articles is messy and unhygienic. The hands or body of the person changing the article can become contaminated due to a failure of the article to contain the exudates or due to exudates that reside on the user's body or garments after removal of the article. In addition, it can be desirable to clean portions of the user's body or garments, which can also lead to contamination of the hands of the person changing the article. Thus, it would be beneficial to have a means for cleaning soiled skin and/or garments while using or changing an absorbent article.

One means for cleaning soiled surfaces is a wipe. Such wipes can include, for example, wet wipes or dry wipes. Wet wipes used for cleansing are often made of a nonwoven material and comprise a liquid cleansing solution. Some wipes contain a fragrance and/or a lotion. Typically, such wet wipes are generally uniform in color and rectangular in shape. Multiple wipes are often packaged together in a common package, but wipes can also be individually packaged.

When cleaning soiled skin while using or changing an absorbent article, the wipe typically will be placed in the user's hand. Generally, the user can prefer that their hand is not contaminated by any body exudates that can be present while the wipe is being used. A rectangular-shaped wipe typically can cover a portion of the user's hand, but can also leave portions of the user's hand uncovered and/or exposed to potential contamination. In addition or alternatively, parts of a rectangular-shaped wipe can extend off the user's hand, potentially leading to waste. It can be awkward and inefficient for the user to receive the maximum benefit from a wipe that does not properly fit the user's hand when changing or using an absorbent article.

As such, it would be desirable to provide a combination absorbent article and wipe product that provides a wipe that is designed to better fit with a user's hand.

### SUMMARY OF THE INVENTION

An absorbent article combination comprising an absorbent article and a pouch joined to the absorbent article, the pouch having a wipe disposed therein, is provided. The wipe can be a functional shape, such as, e.g., a square, an oval, a heart, a square with rounded corners, or a rectangular with rounded corners, or a mitt.

The wipe can be individually packaged, such as, for example, in a pouch, and attached to an absorbent article that can be individually packaged. The wipe can be a wet wipe. A package comprising an absorbent article and a wipe that can be in the shape of a mitt and/or a functional shape is also provided.

### BRIEF DESCRIPTION OF THE DRAWINGS

FIG. 1 is a cross-sectional side view of one embodiment of the present invention.

FIG. 2 is a perspective view of an alternative embodiment of the present invention.

FIG. 3 is a cross-sectional view of one embodiment of the present invention.

FIG. 4 is a perspective view of a wipe in the form of a mitt.

FIG. 5 is a top plan view of the mitt of FIG. 4.

FIG. 6 is a sectional view along 2-2 of one embodiment of the mitt of FIG. 1.

FIG. 7 is a bottom plan view of one embodiment of the present invention.

FIG. 8 is a perspective view of one embodiment of the present invention.

FIG. 9 is a perspective view of one embodiment of the present invention.

FIG. 10 is a perspective view of one embodiment of the present invention.

### DETAILED DESCRIPTION OF THE INVENTION

The present invention relates to functional wipes that can be used in conjunction with an absorbent article. The functional wipes, such as, e.g., wet wipes, can be designed to have a more functional shape as compared to a standard rectangular wipe. The wipes can be designed to better fit a user's hand for an easier and more efficient usage experience. In certain embodiments, a wipe that has a functional shape can better clean the skin while keeping the user's hands clean. For example, the wipes can be square, oval, or heart shaped, generally square with rounded corners, or generally rectangular with rounded corners. In certain embodiments, the wipe can be in the shape of a mitt. The wipe can comprise an inner layer that reduces and/or prevents the user's hand from contacting the body exudates and/or any compositions that the wipe can include. In certain embodiments, the wipe can be individually packaged and can be joined to a sanitary napkin that can be individually packaged.

The combination of a wipe with an absorbent article can provide one or more of several advantages. For example, the wipe can provide for cleansing, which can provide the wearer with increased comfort, physically and/or emotionally. The use of the wipe can also provide a reduction in the odor and/or perceived odor associated with menstruation or other bodily exudates. In addition, the use of the wipe can provide emotional benefits to the user by providing a feeling of freshness and/or cleanliness.

As used herein, the term “absorbent article” refers to devices that absorb and/or contain a substance, such as, for example, body exudates. A typical absorbent article can be placed against or in proximity to the body of the wearer to absorb and contain various exudates discharged from the body.

“Individually packaged,” as used herein, means that only one item is packaged a particular package.

“Absorbent article combination” as used herein refers to an absorbent article with a wipe joined thereto.

As used herein the term “joined” refers to the condition where a first member or component is attached, affixed, or otherwise physically connected to a second member or component either directly or indirectly. An example of an indirectly joined member or component might be where the first member or component is affixed or connected to an intermediate member or component that in turn is affixed or connected to the second member or component. The joined relationship between the first member or component and the second member or component can be fixedly or releasably.

As used herein the term “fixedly joined” refers to a connection that cannot be unattached without at least partially destroying one of the attached components. As used herein the term “releasably joined” refers to a connection that is meant to be easily released.

As used herein, the term “wipe” refers to a piece of material that can be used to clean a user's skin or a surface. The wipe can be made of any suitable material, such as, for example, nonwoven material, paper material, woven material, knitted material, tufted material, stitch-bonded material incorporating binding yarns or filaments, or material felted by wet-milling. Nonwoven material as used herein is “a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling.” “ISO 9092:1988, Textiles-Nonwovens-Definition” International Organization for Standardization, 1988. As such, the wipe can be a “nonwoven wipe,” that is, a wipe comprising a nonwoven substrate. The wipe can also be a “paper wipe,” that is, a wipe comprising a paper substrate, such as, for example, a wet-laid substrate like toilet tissue or facial tissue.

The term “wet wipe” as used herein refers to a wipe that includes a substrate that is moistened with any solution prior to packaging and/or use.

As used herein, the term “mitt” refers to a covering for a user's hand, such as, e.g., a covering having an enclosure that leaves the fingers unseparated and that can include space for the thumb in the main enclosure or that can provide space for the thumb in a separate enclosure for the thumb, or a covering with an enclosure for each finger, such as, e.g., a glove.

As used herein, the term “glove” refers to a covering for the hand having separate sections for each finger.

As used herein, the term “functional shape,” with respect to wipes, refers to a shape that can provide a better fit for a user's hands and/or for an easier and/or more efficient usage experience.

The term “image” when used with reference to printing includes, for example, any type of mark, figure, picture, identification code, symbol, icon, pattern, or text, such as, e.g., a word, nomenclature, number, sentence, or instruction. An image can also be a line, line segment, curved line, band, arrow, area of coloration, or any other printed indicia having a purpose of providing a signal or guide to the user.

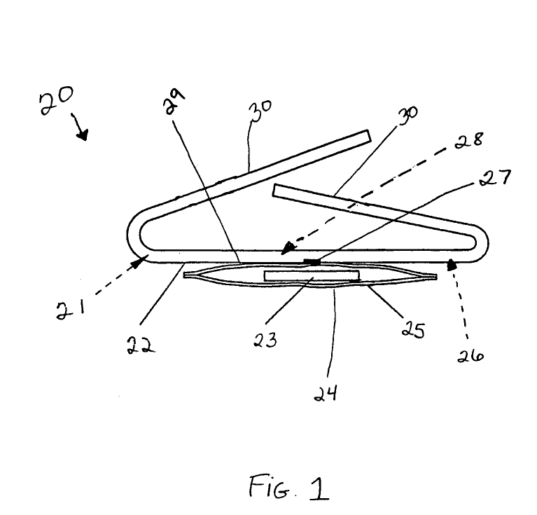

FIG. 1 shows a cross-sectional side view showing an absorbent article combination 20 that comprises a sanitary napkin 21 and an individually packaged wipe 23 that is in a pouch 25. In this embodiment, the pouch 25 is joined to the sanitary napkin 21 by a joining means 27. The pouch 25 can be joined to the sanitary napkin 21 either releasably or fixedly. Any suitable means for joining the pouch 25 to the sanitary napkin 21 can be used. One example of a suitable joining means 27 is an adhesive, such as, e.g., a pressure sensitive adhesive. In certain embodiments, the sanitary napkin 21 and pouch 25 are releasably joined using a glue attachment, such as, for example, hot melt glue. The sanitary napkin 21 can have a releasable wrapper 22 that is either releasably joined to or not joined to the sanitary napkin 21. The releasable wrapper 22 can either partially or completely surround the sanitary napkin 21. In certain embodiments, the pouch 25 can be releasably joined to the releasable wrapper 22 of the sanitary napkin. Also, or alternatively, the pouch 25 can be fixedly joined.

The sanitary napkin 21 shown in FIG. 1 has a body-facing side 28 and an opposed garment-facing side 26. The pouch 25 shown has an outwardly facing side 24 and an opposed inwardly facing side 29. The inwardly facing side 29 of the pouch 25 can be joined to the garment facing side 26 of the sanitary napkin 21. FIG. 1 shows the pouch 25 joined to the sanitary napkin 21 in the middle of the garment-facing side 26. The pouch 25 can be joined to the sanitary napkin 21 at any suitable location on the sanitary napkin 21, the releasable wrapper 22, or a package enclosing the sanitary napkin 21. In certain embodiments, the pouch 25 is joined to the sanitary napkin 21 at one end 30 of the napkin 21. Alternatively, the pouch 25 could be located on the body-facing side 28 of the sanitary napkin 21. These locations can permit the pouch 25 containing the wipe 23 to be removed and used before or without opening the sanitary napkin 21.

In certain embodiments, the sanitary napkin 21 can comprise a pair of flaps that each extend from a longitudinal side margin. Suitable sanitary napkins are described in, for example, U.S. Pat. Nos. 5,346,486; 5,009,653; 4,950,264; 4,917,697; 4,687,478; 4,589,876; and 4,285,343. In such embodiments, the flaps of the sanitary napkin 21 are folded over the topsheet in a topsheet facing relationship, and the pouch 25 containing the wipe 23 is releasably affixed to the flaps such that the topsheet facing relationship is maintained. Such an arrangement is described in, for example, U.S. Pat. No. 6,911,022.

Any pouch 25 suitable for holding a wipe can be used. In certain embodiments, the pouch 25 can be separate from the absorbent article prior to attachment. For example, a pouch separate from an absorbent article may not comprise a component of the absorbent article. In other embodiments, the releasable wrapper 22 can comprise a flap (or pouch 25) to contain a wipe. A suitable flap (or pouch 25) is described in, for example, U.S. Pat. Nos. 4,556,146 and 5,569,230. Alternatively, the absorbent article comprises a releasable paper wrapper 22 that surrounds the absorbent article to form an individual package and the pouch 25 comprises a portion of the releasable paper wrapper 22, such as, for example, the pouches described in, e.g., U.S. Pat. No. 5,569,230.

With respect to the sanitary napkin 21 shown in FIG. 1, the sanitary napkin 21 can be individually packaged, such as, for example, in a flexible poly wrapper. In certain embodiments, the releasable paper wrapper 22 of the sanitary napkin 21 can surround the sanitary napkin 21 to form a package. The individually wrapped sanitary napkins can be packaged in a retail package, such as, for example, a box or bag, such as a poly bag.

Although FIG. 1 shows a sanitary napkin 21 as the absorbent article, any suitable absorbent article can be used, such as, e.g., pantiliners, incontinence articles, interlabial pads, or baby diapers. Suitable pantiliners are disclosed in U.S. Pat. No. 4,738,676. Other absorbent articles are described in, for example, U.S. Pat. Nos. 6,059,764; 5,951,536; 5,824,004; 5,702,382; 5,683,375; 5,658,269; 5,439,458; 5,304,161; and 5,300,054.

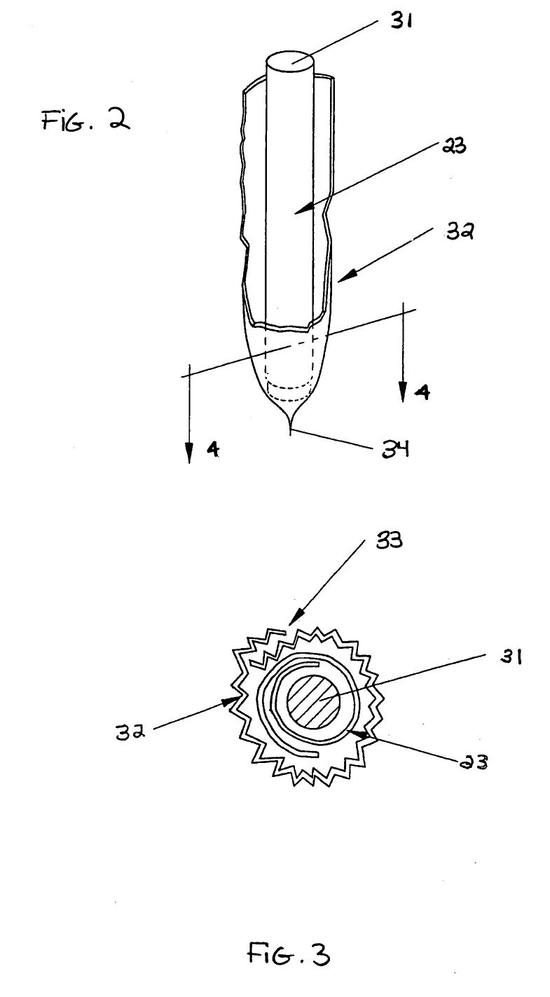

FIGS. 2 and 3 show an example of an embodiment for packaging a wipe 23 with a tampon 31. The tampon 31 can be any known form, such as, for example, tampons disclosed in U.S. Pat. No. 4,475,911. The wipe 23 can be wrapped around the tampon 31. The wrapped tampon 31 can be wrapped with a moisture and vapor impervious overwrap 32. The side 33 and ends (one of which is shown as 34) of the overwrap 32 are sealed. The tampon package can be provided with a releasable seal at the side 33, and/or at least one of the ends 34 for use in opening the package. Any suitable configuration and/or means for joining the tampon 31 and the wipe 23 can be used. For example, in certain embodiments, the personal wipe 23 can be releasably attached to the overwrap 32 of the tampon 31.

In certain embodiments, the wipe 23 can be individually packaged and can be joined to the absorbent article. One individually packaged wipe 23 can be joined to one individually packaged absorbent article, more than one individually packaged wipe 23 can be joined to one individually packaged absorbent article, one individually packaged wipe 23 can be joined to more than one absorbent article that can be individually packaged, more than one wipe packaged together can be joined to one or more individually packaged absorbent articles, one or more individually packaged wipes can be joined to one or more absorbent articles packaged together, or any suitable combination. In certain embodiments, the wipe can be individually packaged, but is not joined to the absorbent article. For example, the wipes can be packaged in individual pouches and a plurality of individual pouches containing wipes can be packaged with absorbent articles in the same exterior packaging or the plurality of individual pouches can be packaged in a different exterior package from that of the absorbent article. The wipes and the absorbent articles can be co-marketed, such as, e.g., designed to be sold and/or used together, such as, for example, for the same purpose. Alternatively, a plurality of wipes can be packaged together in a common package and then packaged with absorbent articles in the same exterior packaging or the plurality of wipes can be packaged together in a common package that is different from that of the absorbent article. In certain embodiments, a plurality of individually packaged wipes are packaged with a plurality of individually packaged absorbent articles wherein the individual package of one or both of the absorbent article or the wipe is provided with an attachment means that allows attachment of the wipe 23 to the absorbent article, such as described in, for example, WO 2005/115295.

In certain embodiments, a plurality of wipes is packaged together. The wipes can be packaged in individual pouches and then packaged in any suitable packaging. Alternatively, the wipes can be packaged together, such as, for example, in a stack, and then packaged in packaging, such as, for example, a hard-sided container or tub; or a soft-sided package such as a pouch 25 or bag.

The wipe 23 can be any size and/or shape suitable for cleansing the skin or providing other benefits when using or changing an absorbent article. In certain embodiments, the wipe 23 can be designed to have a more feminine, more ergonomic, and/or more functional shape as compared to a standard rectangular wipe 23. For example, the wipe 23 can be designed to better fit a user's hand for an easier and more efficient usage experience.

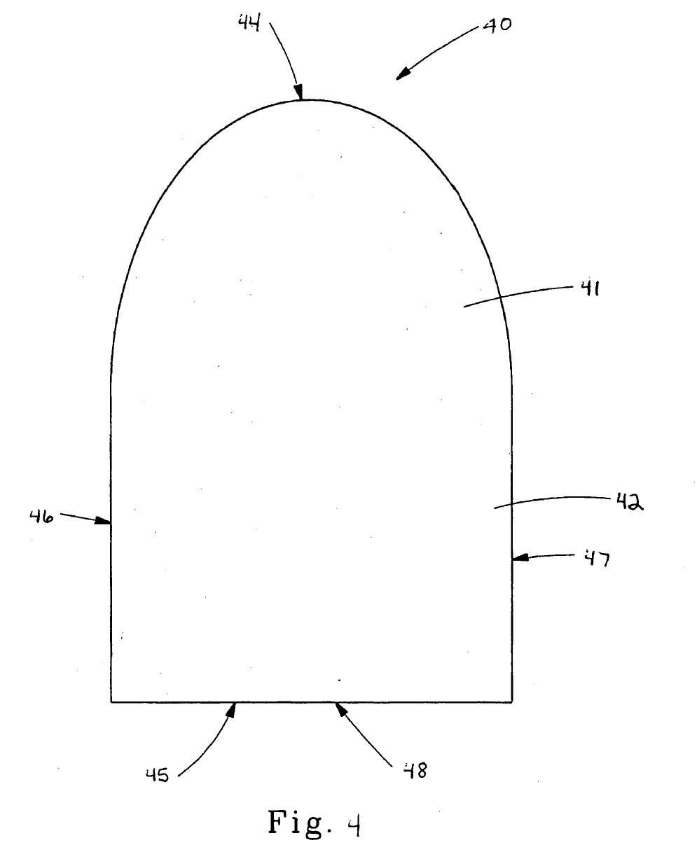

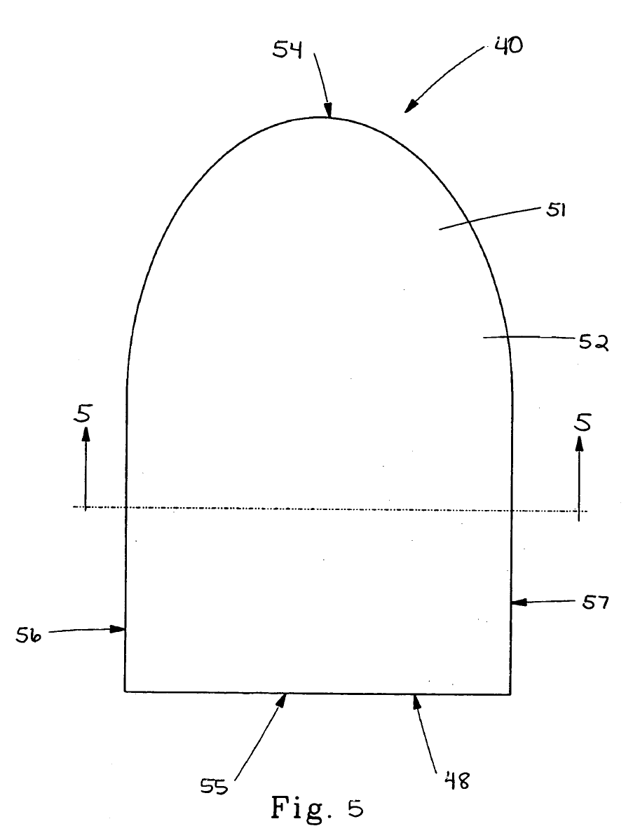

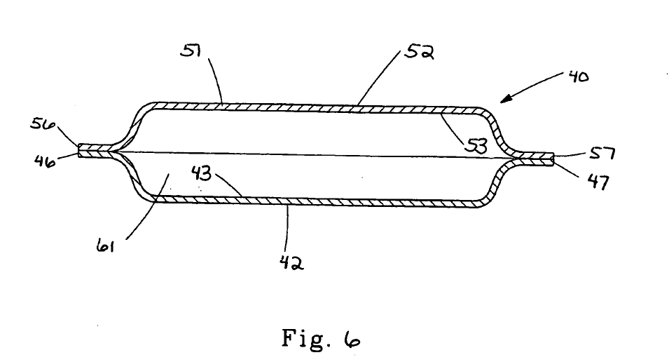

In certain embodiments, the wipe 23 can be in the shape of a mitt, such as, for example, the mitt 40 shown in FIG. 4. The mitt 40 comprises a first sheet 41 that has an exterior surface 42, an interior surface 43 (shown in FIG. 6), a top edge 44, a bottom edge 45, a first side edge 46, and a second side edge 47. The first sheet 41, together with the second sheet 51 (shown in FIG. 5), which are secured in an overlying relationship, define an interior volume 61 (shown in FIG. 6), that is accessed by the user's hand via opening 48. The mitt 40 can also comprise an inner layer or layers, such as, e.g., plastic, film and/or laminate, that reduce and/or prevent the user's hand from contacting the body exudates and/or any compositions the wipe 23 can include. In certain embodiments, the inner layer is a liquid impermeable layer. The wipe 23 can include one or more compositions on one or both sides of the mitt.

FIG. 5 shows a top plan view of the mitt 40 of FIG. 4. The mitt 40 comprises a second sheet 51 that has an exterior surface 52, an interior surface 53 (shown in FIG. 6), a top edge 54, a bottom edge 55, a first side edge 56, and a second side edge 57.

FIG. 6 is a sectional view along 2-2 showing a section view of one embodiment of the mitt 40 shown in FIG. 4.

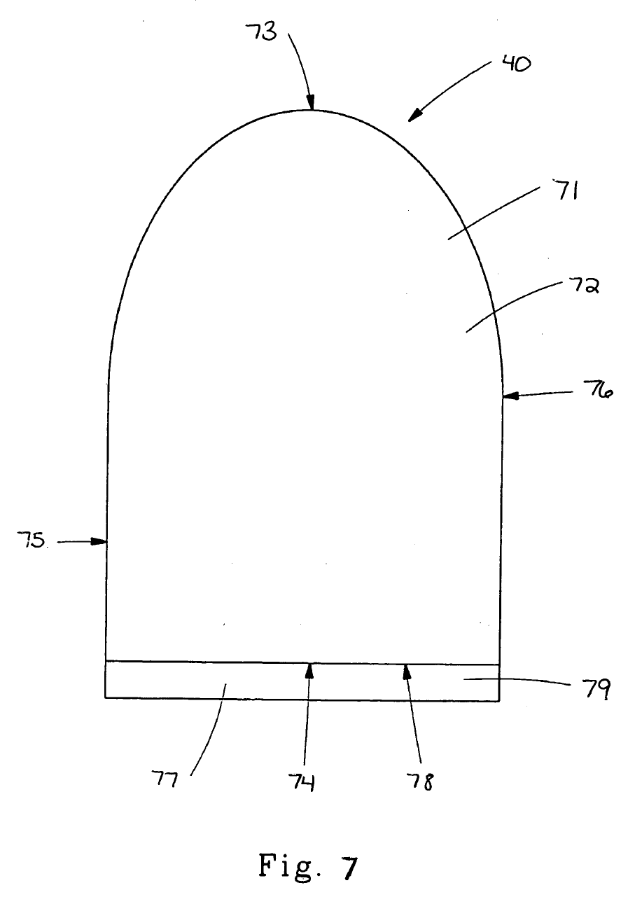

FIG. 7 shows one embodiment of a mitt 40. The mitt 40 comprises a first sheet 71, which has an exterior surface 72, an interior surface, a top edge 73, a bottom edge 74, a first side edge 75, and a second side edge 76. The first sheet 71 can be in an overlying relationship with the substantially complementary second sheet 77. The first sheet 71 and the second sheet 77 can be in an overlying relationship and can define an interior volume that is accessed by the hand via 78. In FIG. 7, the mitt 40 can include a second sheet 77 that is slightly longer than the first sheet 71, giving rise to an extension portion or tab 79. In certain embodiments, the tab 79 can provide a visual and/or tactile clue as to how a user can access the interior volume and/or provide assistance in separating the first sheet 71 and second sheet 77.

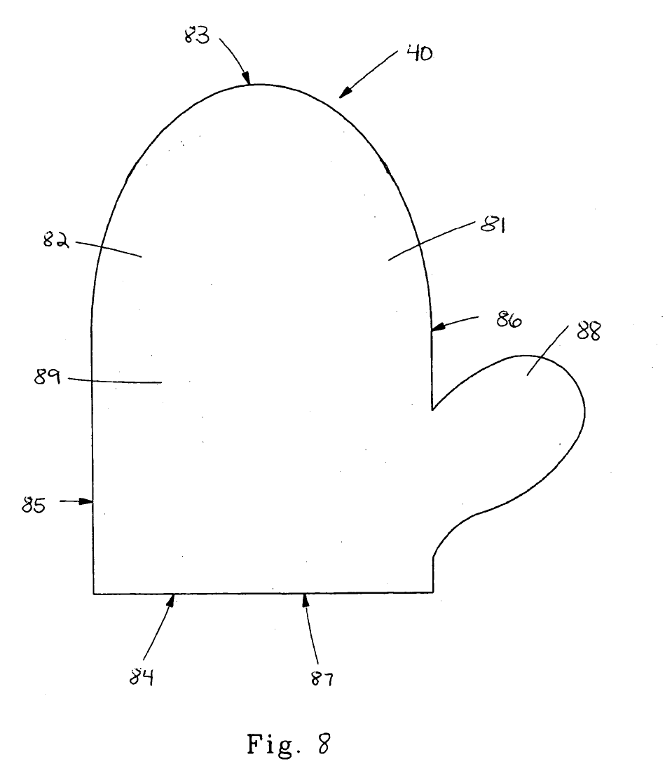

FIG. 8 shows another embodiment of a mitt 40. The mitt 40 includes a first sheet 81, which has an exterior surface 82, an interior surface, a top edge 83, a bottom edge 84, a first side edge 85, and a second side edge 86. The first sheet 81, together with a substantially complementary second sheet, which can be in an overlying relationship, define an interior volume that is accessed by the user's hand via the opening 87. The interior volume of mitt 40 is divided into two parts, one part for the user's thumb 88 and one part for the remainder of the user's hand 89.

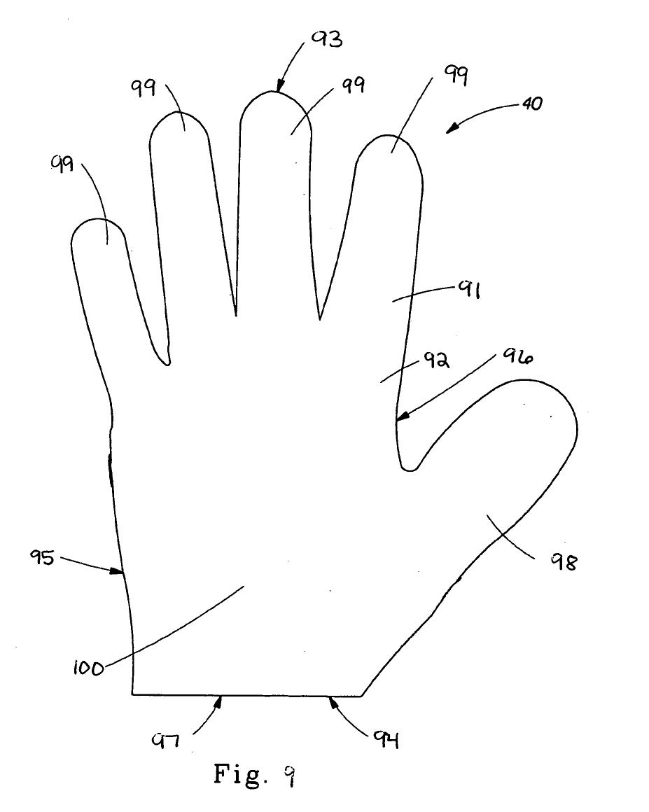

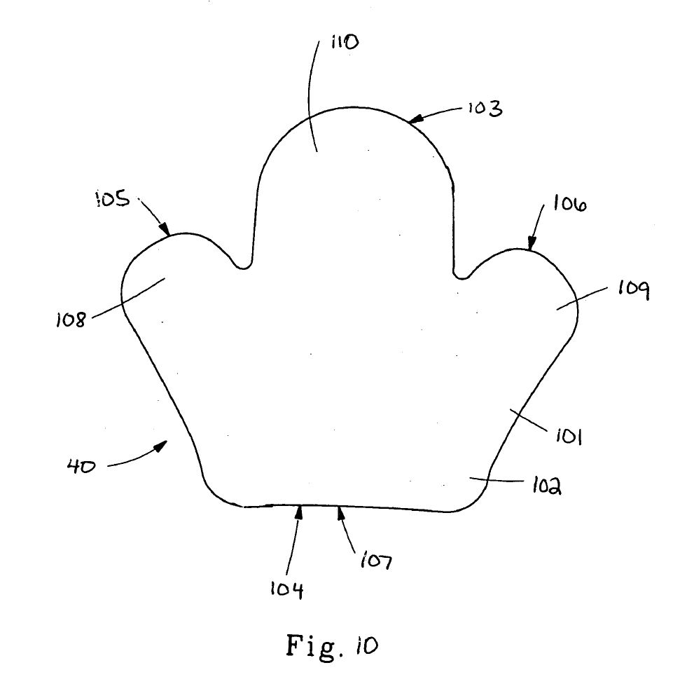

FIG. 9 shows another possible embodiment of mitt 40. The mitt 50 (or glove in this embodiment) includes a first sheet 91, which has an exterior surface 92, an interior surface, a top edge 93, a bottom edge 94, a first side edge 95, and a second side edge 96. The first sheet 91, together with a substantially complementary second sheet, which can be in an overlying relationship, define an interior volume that is accessed by the user's hand via opening 97. The interior volume of mitt 40 is divided into several parts, one part for the user's thumb 98, individual sections for each of the user's fingers 99, and one part for the remainder of the user's hand 100.

The mitts illustrated in FIGS. 8 and 9 are “right-handed” mitts. That is, the mitts of FIGS. 8 and 9 can be worn on the user's right hand. The mitt can also be a “left-handed” mitt that can be worn on the user's left hand. In certain embodiments, such as shown in FIGS. 4-7, the mitt can be “thumbless,” i.e., having a single monolithic or undivided interior volume. Alternatively, the mitt can include two opposing thumb portions, thereby enabling the mitt to be used equally on either the user's right or left hand. The mitt can be the same on both sides such that the mitt can be used on either hand.

FIG. 10 illustrates a mitt 40 that includes two opposing thumb portions, thereby allowing the same mitt to be used on either hand. The mitt 40 comprises a first sheet 101, which has an exterior surface 102, an interior surface, a top edge 103, a bottom edge 104, a first side edge 105, and a second side edge 106. The first sheet 101, together with a substantially complementary second sheet, which are in an overlying relationship, define an interior volume that can be accessed by the user's hand via opening 107. The interior volume of mitt 40 is divided into several parts, two opposing thumb portions 108 and 109, and one part for the remainder of the user's hand 110.

In certain embodiments, the wipe 23 can be generally square in shape. For example, the wipe 23 can have four sides that are at least about 85%, at least about 90%, at least about 95% the same in length. In certain embodiments, the wipe 23 can be generally square in shape with rounded corners. In certain embodiments, the wipe 23 can be generally oval, generally heart-shaped, and/or generally round in shape. The wipe 23 can have any dimensions suitable for cleansing the skin and/or garments and/or for providing other benefits. In certain embodiments, the wipe 23 can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size.

In certain embodiments, the wipe 23 can have at least one region, edge, and/or periphery that can be non-linear. When cutting a first wipe 23 having at least one region, edge, and/or periphery that can be non-linear and a second wipe 23 having at least one region, edge, and/or periphery that can be non-linear from a larger piece of substrate, it can be desirable to align one or more of the regions, edges, and/or peripheries of the first wipe and second wipe together when cutting the first wipe and second wipe from the substrate, for example, such that waste is reduced. Thus, in certain embodiments, when cutting a wipe 23 having at least one non-linear region from a web, the wipe 23 can have a region that adjoins the edge of the previous wipe cut from the web.

The wipe 23 can be printed. In certain embodiments, the wipe 23 can be embossed. At least one or all of the embossed depressions can have therein a substance such as ink to provide visible color, for example, as described in WO 04/057110, or U.S. Pat. No. 6,780,270. The color can also be on a separate layer, such that upon embossing, color shows through the embossed portion, thereby giving the appearance of having been printed in registration with the embossment.

The wipe 23 can be a nonwoven wipe 23 comprising a substrate comprising a nonwoven web of natural fibers, synthetic fibers, or mixtures of natural and synthetic fibers wherein the substrate is not considered paper. Suitable natural fibers include, e.g., cellulosic fibers, such as wood pulp fibers, cotton, and rayon. Suitable synthetic fibers include fibers commonly used in textiles, such as, for example, polyolefins, such as polyester and polypropylene fibers. In certain embodiments, the wipe 23 is a nonwoven wipe 23 comprising a substrate that comprises viscose rayon and/or wood pulp. Alternatively, the wipe 23 can be a paper wipe, such as, e.g., a wipe 23 comprising a substrate that is made of a wet-laid material in a manner similar to toilet tissue or facial tissue. In certain embodiments, the wipe can comprise silk.

The wipe 23 can be “flushable”, that is, it is of a size of is capable of disintegrating to pieces sufficiently small such that when flushed in a toilet and when being transported in the sewer system, it does not plug any element of a sewer system. Any wrapping material associated with the wipe 23, such as, for example, the pouch 25 material, can also be flushable. Wrapping materials that meet such criteria can include, for example, water soluble materials, such as, e.g., silicone-treated polyvinyl alcohol films, films coated with a polyvinyl alcohol, and tissue coated or impregnated with polyvinyl alcohol. Suitable materials are disclosed in, e.g., U.S. Pat. No. 4,755,421.

The wipe 23 can be a wet wipe that comprises a liquid-based cleansing solution. The wipe 23 can also, or alternatively, be scented. In certain embodiments, the wipe 23 can comprise a nonwoven fabric that includes a cleansing solution. The cleansing solution can include, for example, surfactants, alcohols, fragrance compositions, antimicrobial agents, pH buffers, and combinations thereof. The wipe 23 can also contain substances such as silicones that can inhibit body exudates from adhering to the user's body. The cleaning solution can also contain skin conditioning substances similar to those used in hand lotions, such as, e.g., aloe and Vitamin E; other substances for applying to the skin, such as, e.g., witch hazel; or any other substances known in the art for inclusion in wet wipes.

The wipe 23 can comprise a lotion, such as, for example, an aqueous or non-aqueous lotion. In certain embodiments, the lotion can increase the cleaning capabilities of the wipe 23 by aiding removal of soiling from the skin. The lotion can also provide skin benefits by reducing friction between the skin and the wipe 23 and/or by containing materials that can soothe the skin, such as, for example, aloe or chamomile. Examples for suitable lotions are disclosed in, for example, EP 808151; EP 763,341; and WO 00/57843.

One or more fragrance compositions can be included in the cleansing solution in order to produce a scented wipe 23. Alternatively, the one or more fragrance compositions can be included in the wipe 23 separately from any cleansing solution such as, for example, by applying the fragrance composition to the wipe 23 prior to, after, or instead of any cleansing solution that can be added. Another example might be a wipe 23 that includes a separate component such as, for example, a carrier material comprising a fragrance composition. Additionally, the one or more fragrance compositions can be disposed together or separately on at least a portion of the wipe 23.

Other embodiments can include dry wipes or wipes containing non-aqueous cleaning solutions such as, for example, mineral oils, and the emollient described in U.S. Pat. No. 4,481,243.

In certain embodiments, the wipe 23 can be packaged in a pouch 25. Any suitable pouch 25 for holding a wipe 23 can be used. For example, the pouch can be made of paper and/or other suitable materials, such as, e.g., polyethylene. When the wipe 23 is a wet wipe, any pouch 25 suitable for maintaining the wetness of the wipe 23 can be used. For example, the pouch 25 can comprise at least one barrier material suitable for preventing evaporation of moisture from the wipe 23 can be used. Suitable barrier materials include, for example, laminates with a layer of ethylene vinyl alcohol (EVOH), propylene vinyl alcohol (PVOH), or aluminum sandwiched between polymeric film layers, such as LDPE (low density polyethylene); metallized, such as aluminized plastic films; metal foils; oriented polyethylene terephtalate (PET); PETG (glycol-modified PET); oriented polyamide; aromatic polyamide; or polymeric films like polyethylene films. In certain embodiments, the pouch 25 comprises paper and/or foil. Also, or alternatively, the pouch 25 can comprise an inner layer comprising polyethylene (PE), an outer layer comprising a copolymer of polyethylene (PE) and polypropylene (PP) and a subjacent intermediate layer comprising polypropylene (PP).

In certain embodiments, one or more sides of the pouch 25 can be sealed to prevent substantial evaporation of the liquid solution from the time the product is manufactured to the time it is used by the consumer. Any number of sealing mechanisms can be used including, for example, heat and/or pressure seals, ultrasonic seals, glue seals, zippered track sealing systems such as Dow Company's “ZIP LOCKS” or combinations thereof. In certain embodiments, the pouch 25 can be hermetically sealed. In addition, the pouch 25 can be lined with foil, or other suitable material to further reduce evaporation.

The pouch 25 can include a means for unsealing (or opening) the pouch 25 in order to access a wipe 23 contained therein. For example, the pouch 25 can have a scored or die cut line of weakness to allow the pouch 25 to be opened easily with a tab, easy peel label or any other opening mechanism; a “Dry-edge” sticker mechanism, such as, e.g., a sticker with a dry edge for grasping; releasable adhesives; releasable heat and/or pressure seals; perforations; rupture seals; tear seals; or the pouch 25 can be made of a relatively easy to tear material such as, for example, an easy to tear film.

Any suitable joining means can be used to attach the pouch 25 and absorbent article of the absorbent article combination 20. In certain embodiments, the joining means on the outside of the package of the wipe 23 can be selected and applied such that a sufficient degree of joining between the package of the absorbent article and the pouch 25 of the wipe 23 is generated. Any suitable adhesive can be used to join the absorbent article and the pouch 25, such as, for example, a pressure-sensitive adhesive, hot melt adhesive, cold melt adhesive, or solvent-based adhesives. The adhesive can be applied to the absorbent article and/or pouch 25 as a continuously coated area or as a pattern, such as, e.g., dots, stripes, spirals, or beads. In certain embodiments, the absorbent article and the pouch 25 can be joined by one or more mechanical fasteners. Suitable mechanical fasteners include, for example, hook and loop fasteners.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article combination comprising:
a. an absorbent article; and
b. a pouch joined to the absorbent article, the pouch having a wipe disposed therein, wherein the wipe is in the shape of a mitt.

2. The absorbent article combination of claim 1, wherein the mitt comprises first and second sheets secured in an overlying relationship, the members defining an interior volume for receiving a user's hand.

3. The absorbent article combination of claim 2, wherein the mitt comprises a liquid impermeable inner layer.

4. The absorbent article combination of claim 1, wherein the mitt is thumbless.

5. The absorbent article combination of claim 1, wherein the mitt is a glove.

6. The absorbent article combination of claim 2, wherein the interior volume includes space for the user's fingers and wherein the mitt comprises a space for the user's thumb in a separate enclosure for the thumb.

7. The absorbent article combination of claim 1, wherein the pouch comprises a periphery, wherein the periphery is closed.

8. The absorbent article combination of claim 1, wherein the wipe is a wet wipe.

9. The absorbent article combination of claim 1, wherein the absorbent article comprises a releasable wrapper wherein the releasable wrapper at least partially surrounds the absorbent article, and wherein the pouch is joined to the releasable wrapper.

10. An absorbent article combination comprising:
a. an absorbent article; and
b. a pouch joined to the absorbent article, the pouch having a wipe disposed therein, wherein the wipe is a functional shape.

11. The absorbent article combination of claim 10, wherein the functional shape is a square, an oval, a heart, a square with rounded corners, or a rectangular with rounded corners.

12. The absorbent article combination of claim 10, wherein the pouch comprises a periphery, wherein the periphery is closed.

13. The absorbent article combination of claim 10, wherein the wipe comprises a nonwoven material.

14. The absorbent article combination of claim 10, wherein the wipe is a wet wipe.

15. The absorbent article combination of claim 10, wherein the absorbent article comprises a releasable wrapper wherein the releasable wrapper at least partially surrounds the absorbent article, and wherein the pouch is joined to the releasable wrapper.

16. A package comprising:
a. an absorbent article; and
b. a wipe, wherein the wipe is a functional shape.

17. The package of claim 16, wherein the wipe and/or absorbent article is individually packaged.

18. The package of claim 16, wherein the functional shape is a square, an oval, a heart, a square with rounded corners, a rectangular with rounded corners, or a mitt.

19. A method of cutting two or more wipes from a substrate, the method comprising: cutting a first wipe having at least one region, edge, and/or periphery that is non-linear from a substrate and cutting a second wipe having at least one region, edge, and/or periphery that is non-linear from the substrate, wherein the second wipe has a region that adjoins a region of the first wipe.

20. The method of claim 19, wherein at least one of the first wipe and second wipe is a functional shape.
